(19) United States
(12) Patent Application Publication
(10) Pub. No.: US 2006/0003452 A1
(43) Pub. Date: Jan. 5, 2006

(54) Vector packaging cell line

(75) Inventors: Laurent Humeau, Germantown, MD (US); Vladimir Slepushkin, Damascus, MD (US); Brian Paszkiet, Frederick, MD (US); Yajin Ni, Germantown, MD (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: VIRxSYS Corporation, Gaithersburg, MD (US)

(21) Appl. No.: 11/172,147

(22) Filed: Jun. 30, 2005

Related U.S. Application Data
(60) Provisional application No. 60/585,464, filed on Jul. 1, 2004.

(51) Int. Cl. C12N 15/867; C12N 5/08

(52) U.S. Cl. 435/456; 435/372

(57) ABSTRACT

The invention relates to a method of increasing vector transduction in target cells. The invention provides for the recombinant engineering of a packaging cell line to be capable of expressing one or more membrane proteins which facilitate binding to, and activation of, a target cell. The invention also provides for recombinant engineering of a cell that endogenously expresses one or more such membrane proteins into a packaging cell line. A vector packaged into viral particles via use of such cell lines would comprise an outer envelope containing these proteins. The particles would be specifically suited for binding and targeting to a target cell to facilitate transduction thereof with the vector. The target cell may also be simultaneously activated (stimulated) by the packaged vector in the absence of exogenously supplied stimulatory molecules.

### RELATED APPLICATIONS

This invention claims benefit of priority from provisional U.S. Patent Application 60/585,464, filed Jul. 1, 2004, which is hereby incorporated by reference as if fully set forth.

### FIELD OF THE INVENTION

This invention relates to a method of increasing vector transduction in target cells. The invention provides for the recombinant engineering of a packaging cell line to be capable of expressing one or more membrane proteins which facilitate binding to, and activation of, a target cell. Alternatively, the invention provides for recombinant engineering of a cell that endogenously expresses one or more such membrane proteins into a packaging cell line. A vector packaged into viral particles via use of such cell lines would comprise an outer envelope containing these proteins. The particles would be specifically suited for binding and targeting to a target cell to facilitate transduction thereof with the vector. The target cell may also be simultaneously activated or stimulated by the packaged vector in the absence or minimal presence of exogenously supplied stimulatory molecules.

### BACKGROUND OF THE INVENTION

Lentiviral vectors have been shown to transduce classically non-dividing cells such as neurons, and the ability of these vectors to deliver genes to target cells in the absence of cell division is one of the defining features and advantages of lentivirus-based vectors (Naldini et al, 1996-1; Naldini et al, 1996-2). However, driving a population of dividing cells into activation in a coordinated manner facilitates simultaneous transduction of the whole cell population at high levels (Humeau et al, 2004; Park et al, 2000). Transduction efficiencies in T cells with the first lentiviral vectors ranged from 20-40% transduction (Schroers et al, 2002; Costello et al, 2000; Ranga et al, 1998; Mitsuasu et al, 2000), which were significantly raised as high as 60-75% transduction with the addition of the cPPT/CTS sequence to increase the rate of nuclear translocation of the proviral sequence (Manganini et al, 2002; Follenzi et al, 2000; Cavalieri et al, 2003).

These previous reports have used methods of transduction involving either spinoculation to concentrate the vector with the target cell to increase the chance of gene transfer or prestimulation of the cells for usually three days followed by single or multiple vector additions (Levine et al., 1997; Levine et al., 2000). Such methods are difficult to use in a clinical setting where the culture system is closed and sequential manipulations are difficult. The most recently developed method of transduction involves the simultaneous addition of vector, cells, and the T cell stimulatory molecules CD3 and CD28 to drive the cells into division (Lu et al, 2004; Humeau et al, 2004). Exposure to CD3 and CD28 has been previously shown to increase transgene expression when used to prestimulate the cells prior to vector addition (Costello et al, 2000). This combined transduction and culture initiation procedure results in maximum transduction efficiencies of 99% and reduces the time required to achieve such levels to three days (Lu et al, 2004). These studies underscore the important role of cell stimulation in reaching transduction efficiencies high enough for therapeutic benefit.

Reduced culture time required for transduction also aids in preservation of the natural potency of the cells being transduced. A unique quality to lentiviral vectors is that they do not require cell division for genomic insertion. This is helpful for quiescent cellular targets of gene therapy, which include macrophages, primitive hematopoietic progenitor cells, and circulating naive T lymphocytes, which may be targeted by the instant invention as provided in greater detail below. Such targets are especially valuable therapeutically since they retain exceptional potency for long-term function in the body. Extensive stimulation ex vivo with cytokines or other stimulating molecules can result in a change in their potency in vivo (Maurice et al, 2002; Soares et al, 1998). Therefore, when using an HIV-based vector, inducing a transient switch from G₀ to G_{1b} in quiescent T cells can facilitate vector integration without promoting expansion that results in a loss of the naive profile (Dardalhon et al, 2000). Similarly in hematopoietic stem cells, minimal stimulation will promote efficient gene transfer without inducing a loss in hematological reconstitution potential.

Although recently developed transduction methods in the field represent a significant improvement over earlier methods, the requirement for exogenous costimulatory molecules for cellular activation ex vivo is contrary to the development of a gene therapy vector that may be injected into the body to facilitate efficient transduction in vivo. It is uncertain as to the ability to successfully provide in vivo co-administration of stimulatory molecules with a vector as a therapeutic regimen. Additionally, and even in cases of ex vivo gene transfer applications, the requirement for exogenous costimulatory molecules in the culture raises additional quality control and safety issues.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

This invention provides improved packaging and producer cell lines as well as compositions and methods comprising them to improve retroviral and lentiviral transduction and targeting. In some embodiments, the invention is directed to lentiviral transduction systems that provide lentiviral particles with improved binding and stimulatory properties to facilitate transduction and proliferation of cells targeted by the particles. In other embodiments, the invention provides means to transduce non-dividing cells without solely relying upon high titers of viral particles and/or stimulation with exogenously supplied stimulatory factors. The invention thus has advantages in being used ex vivo or in vitro without the need for soluble stimulatory factors or factors provided by accessory cells. Additionally, the invention may be used in vivo as a direct injectable to deliver vector or viral particle borne “payloads” to a subject.

In a first aspect, the invention provides a way to improve lentiviral transduction and targeting by the incorporation, into the vector envelope, of molecules that possess the ability to bind a target cell and/or stimulate cell-cycle transition of said cell. The ability to stimulate cell-cycle transition has been shown to increase transduction and is the function of CD3 and CD28 ligands in some in vitro and ex vivo transduction protocols. For example, anti-human CD3/CD28 conjugated beads used in T cell transduction have been used by others in the field (Lu et al., 2004 and Levine et al., 1997 and 2000). Other groups have engineered retroviral vectors containing recombinant envelope proteins that mimic costimulatory molecules such as IL-2 and IL-7 (Maurice et al., 1999 and Verhoeyen et al., 2003) or a single chain anti-CD3 antibody (Maurice et al., 2002). The instant invention is based in part on the recognition that incorporation of cell surface proteins and molecules into the envelope surface of a retroviral vector will modulate transduction efficiencies in the absence of exogenous costimulatory molecules.

The invention provides the novel concept of producing a lentiviral vector containing an envelope optimized for attachment to and subsequent transduction, or gene transfer, in a target cell of choice. This is achieved not by producing vectors containing recombinant envelope proteins (Verhoeyen et al., 2003; Maurice et al., 1999 and 2002), but rather by the design and use of a producer cell line engineered to express membrane associated proteins of interest on the vector surface. Vector subsequently produced in this cell line would acquire the membrane-associated proteins, which have the ability to bind to and activate the target cell, and hence be optimized for gene transfer into such cells. Such envelope proteins may be naturally occurring in the producer cells or other cells and include CD49d, CD54, CD80, and CD86, individually or in combinations of two, three or all four, as non-limiting examples.

The invention thus reduces or removes the need to add exogenous molecules for costimulation, and beneficially provides transduced cells in the absence of exogenously provided factors. This permits an expansion in the possible applications and uses of the transduced cells. The invention also yields a simplified and more clinically appropriate approach for ex vivo transduction as well as in vivo gene delivery. The invention may also be used to reduce complications during transduction of a target cell with a packaged particle.

As one exemplary embodiment, lentivirus vectors may be engineered to contain an envelope that mimics as much as possible the natural HIV envelope budding out of its natural cellular hosts and differing only in the viral envelope protein. By including proteins such as, but not limited to, CD86 and CD54, alone or in combination, in these vector particles, the particles may be advantageously used for the stimulation of a target CD4 T cell (via CD86) and/or to increase the binding of a vector to a cell via cell-vector interactions (via CD54).

Thus, one may envision the packaging of vector by use of cells that naturally bind to the target cell in vivo. For example, T cells contain molecules that bind to and activate other T cells in lymphoid tissues. Therefore, T cells or associated T cell lines may be used directly, or after modification, as a packaging cell and then vector producer cell to produce vector intended to bind to T cells. Another example would be using mesenchymal stem cells (MSC) for the production/packaging of vector intended to target and transduce hematopoietic progenitor cells, since MSC provide the supporting matrix for these cells in vivo.

In addition to the possible use of non-modified cells, the invention provides for a recombinant retroviral packaging cell comprising a first nucleic acid molecule that expresses, or is capable of expressing, at least one membrane associated non-viral ligand in said cell. The ligand is non-endogenous (or heterologous relative to said cell or otherwise not normally expressed in said cell) in some embodiments, although a recombinant cell that comprises a nucleic acid construct that increases an endogenous membrane associated non-viral ligand may also be used in the practice of the invention. The non-viral ligand may be a naturally occurring cell surface molecule and is one which binds a cell surface molecule of a target cell. This first nucleic acid molecule may be transiently introduced into said cell or previously stably introduced into the cell. Stably introduced molecules are included embodiments of the invention.

In some embodiments, the at least one membrane associated non-viral ligand plays a role in cell-cell adhesion via binding to said cell surface molecule and/or acts after binding a cell surface molecule of a cell to activate or stimulate the cell into cell cycle transition, leading to or not, to growth and/or proliferation. In some embodiments of the invention, the cell comprises at least two such ligands, such as one which plays a role in cell-cell adhesion and one which activates or stimulates cell cycle transition. In other embodiments of the invention, the ligand is a co-stimulatory molecule that binds a T cell surface molecule to activate T cell proliferation when the CD3/TCR complex of said T cell is bound by a natural or artificial ligand or by a specific antibody (Ab). Other non-limiting ligands are those found on a hematopoietic cell.

The recombinant as well as packaging characteristics of a packaging cell may indicate that the cell comprises at least one heterologous nucleic acid molecule which expresses, or is capable of expressing, one or more viral factor(s) necessary for the packaging of a retroviral vector. The one or more factor(s) are those that function in trans to permit packaging of a retroviral nucleic acid into a virion or viral particle. In some embodiments, the factor(s) are one or more viral structural proteins, such as matrix, capsid, or nucleocapsid proteins, and/or one or more viral accessory proteins. As recognized by those skilled in the art, a packaging cell with a vector to be packaged may be viewed as a packaging system where the packaging components needed in trans may be expressed by a combination of nucleic acid sequences from the packaging cell and from the vector. The at least one heterologous nucleic acid molecule may be transiently introduced into said cell or previously stably introduced into the cell. Stably introduced molecules are included embodiments of the invention.

The viral factor(s) may support the packaging of a lentiviral vector. Any one or a combination of multiple factor(s) may be encoded by nucleic acid sequences in the packaging cell, with any factors not so encoded being encoded by nucleic acid sequences of the vector. In some embodiments, factors include viral envelope proteins and other trans factors, such as the GAG or POL proteins as well as the various accessory proteins in the case of lentiviral vectors. In additional embodiments, packaging cells of the invention express or are capable of expressing a viral envelope protein capable of packaging a lentiviral vector to produce a particle capable of transducing a target cell of interest. In other embodiments, packaging cells express, or are capable of expressing, a viral ligand which binds a cell surface molecule of a target cell.

In one exemplary embodiment of the invention, the viral factor is a protein that retains or mediates fusion of a viral or vector particle with a target cell membrane. Stated differently, the factor functions to mediate fusion of a cell membrane containing said viral ligand with the cell membrane of a target cell. Non-limiting examples include factors that target or bind nectin-1, such as the wild type herpes simplex virus (HSV)-1 envelope protein. Viral and non-viral factors, such as those that target or bind CD34+, CD33+, and/or CD14+ cells, such as the CD226 (DNAM-1) proteins that target the CD155/PVR or CD112 (Nectin-2 present on CD34+ cells), are additional non-limiting examples.

The recombinant as well as packaging characteristics of a packaging cell may also indicate that the cell produces no viral particles in the absence of a vector sequence to be packaged. The vector sequence may be viewed as a second nucleic acid molecule in such a cell, and vectors derived from a lentivirus, which have the long terminal repeat (LTR) regions and/or a lentiviral packaging or dimerization signal are embodiments of the invention.

In further embodiments, target cells of the invention include, but are not limited to, antigen presenting cells (APCs), cells of the hematopoietic lineage, stem cells, lymphocytes, (including T cells and B cells, such as those in the germinal center of a lymph node), neurons, endothelial cells, tumor cells, dendritic cells, fibroblasts, and non-hematopoietic stem cells.

The membrane associated non-viral ligand(s) of the packaging cells, after incorporation into a vector envelope, are used to direct the virion particles to such target cells. The ligands are transmembrane proteins in some embodiments, but they may also be proteins on the outer surface of the packaging cell membrane. The ligand(s) may be those of a higher eukaryotic cell, a synthetic or chimeric (fusion) molecule based thereof, a single chain antibody or binding fragment thereof, or a microbial protein.

The invention also provides for compositions comprising the packaging cells disclosed herein. Such compositions include a combination of a packaging cell with a vector to be packaged. The vector may have been introduced into said cell. In some embodiments, vectors of the invention are those that have been depleted of accessory protein encoding nucleic acids, such as, but not limited to, lentiviral vectors that lack functional sequences capable of expressing lentiviral accessory proteins. Such proteins may be provided in trans via the packaging cell. Other compositions include combinations of the packaging cells with suitable media and/or incubation devices for the propagation of said cells.

In a second aspect, the invention provides for methods of producing packaging cells of the invention. Such methods comprise introduction of a nucleic acid molecule which expresses, or is capable of expressing, a viral gene product necessary for packaging and/or replication of a retrovirus as described herein in said packaging cell; and introduction of at least one nucleic acid molecule, expressing or capable of expressing, in said packaging cell, at least one membrane associated non-viral ligand(s) as described herein.

In a third aspect, the invention provides for methods of producing, or packaging, viral vector particles of the invention. Such methods comprise introduction of a nucleic acid molecule encoding or comprising a retroviral vector into a recombinant packaging cell as described herein. Such a cell is then cultured under conditions wherein said cell packages said vector into a particle comprising at least one membrane associated non-viral ligand(s) as described herein.

In a fourth aspect, the invention provides a retroviral vector packaging cell that is made recombinant by introduction of a nucleic acid molecule encoding a viral packaging factor as described herein. The cell is one that endogenously expresses at least one membrane associated ligand capable of binding to a cell surface molecule of a target cell or tissue. In some embodiments, the cells are those that are non-adherent (e.g. may be propagated in suspension) and/or which interact or bind with the target cell or tissue in vivo. Non-limiting embodiments include bone marrow stromal cells that package vectors for use with hematopoietic stem cells as the target cells. Alternatively, the cell may be one which expresses an integrin protein which binds to the surface of CD34+ cells. In other embodiments, the packaging cell may express a ligand selected from SDF-1, VLA-4, VLA-5, or LFA-1, and the packaged vector thus binds hematopoietic stem cells that are a) CD34+ and CD38−/CXCR4+ or b) CD34+ and CD38 low/CXCR4+.

The invention also provides for compositions comprising such packaging cells, optionally with vectors as described herein, as well as methods of preparing such cells and methods of using such cells to package vectors as disclosed herein.

In a fifth aspect, the invention provides vector containing viral particles produced by the cells, compositions, and methods of the invention. Such particles will have an exterior that contains ligands and other molecules from the packaging cells as described herein.

### DEFINITIONS

The term “recombinant” as used herein refers to the use of genetic engineering and/or molecular biology techniques to produce a biological product such as a protein or nucleic acid. In the case of a recombinant nucleic acid molecule that may be expressed, the term usually refers to a non-naturally occurring molecule that contains a sequence not found in nature. Such a molecule can, of course, be used to express a biological molecule that is naturally occurring or synthetic. In the case of a recombinant cell, the term usually refers to a cell that expresses, or is capable of expressing, a non-endogenous protein or a cell that expresses, or is capable of expressing, a non-endogenous level of an endogenous protein.

The terms “viral particle” or “vector particle” or “virion” or variations thereof refer to a macromolecular complex that normally encloses or contains a viral or vector nucleic acid as described herein. Such particles are normally encased in a lipid bilayer membrane obtained during production by the producer and packaging cells and methods of the instant invention.

### BRIEF DESCRIPTION OF THE DRAWINGS

FIG. 1 shows the ability to increase gene transfer (transduction levels) in primary human CD4+ T lymphocytes by use of a viral vector packaged in cells expressing CD54. HIV based lentiviral vectors were produced in cells expressing or not expressing the CD54 protein in the membrane. Vectors were compared for gene transfer by evaluation of copy number using TaqMan™ based PCR mediated detection in target cells after transduction in the presence or absence of retronectin. n=3.

FIG. 2 shows the cellular growth curve of CD4+-enriched T cells isolated from normal human apheresis, after exposure to one of three conditions: media alone (nada), soluble anti-CD3 antibody (CD3) or anti-CD3/28 antibody-coated beads (B). Cells from each group of three conditions were exposed to one of 5 different vector preparations at a multiplicity of infection (MOI) of 20; vector buffer alone (NV), unmodified control vector (CV), or vector packaged in cells expressing CD54 on the membrane surface (54) or CD86 on the membrane surface (86) or both (54/86). Cells were monitored using a Z2 coulter counter for expansion during a 21-day period. n=3.

FIG. 3 shows the cellular growth curve of CD4+-enriched T cells isolated from normal human apheresis after exposure to one of three conditions: media alone (nada), soluble anti-CD3 antibody (CD3) or anti-CD3/28 antibody-coated beads (B). Cells from each group of three conditions were exposed to one of 5 different vector preparations at an MOI of 20; vector buffer alone (NV), unmodified control vector (CV), or vector produced by transient transfection in 293F cells in combination with CD54 (54) or CD86 (86) or both (54/86). Cells were monitored using a Z2 coulter counter for expansion during a 21-day period. n=3.

FIG. 4 shows the enhancement of activation of primary CD4+ enriched T cells isolated from normal human apheresis product, as measured by increased expression of the activation marker CD25, after co-culture with 293 cells expressing CD86 and CD54 on its membrane surface when compared to CD54 alone. Cells were incubated in the absence of stimulatory anti-CD3 antibody, or a range of anti-CD3 antibody concentrations ranging from 5 μg/ml to 20 μg/ml. CD25 expression (frequency and mean fluorescence) were measured at day 4 post culture initiation. n=3, and standard error from the mean is shown.

FIG. 5 shows the enhancement of activation of primary CD4+ enriched T cells isolated from normal human apheresis product, as measured by increased expression of the activation marker CD69, after co-culture with 293 cells expressing CD86 and CD54 on its membrane surface when compared to CD54 alone. Cells were incubated in the absence of stimulatory anti-CD3 antibody, or a range of anti-CD3 antibody concentrations ranging from 5 μg/ml to 20 μg/ml. CD69 expression (frequency and mean fluorescence) were measured at day 4 post culture initiation. n=3, and standard error from the mean is shown.

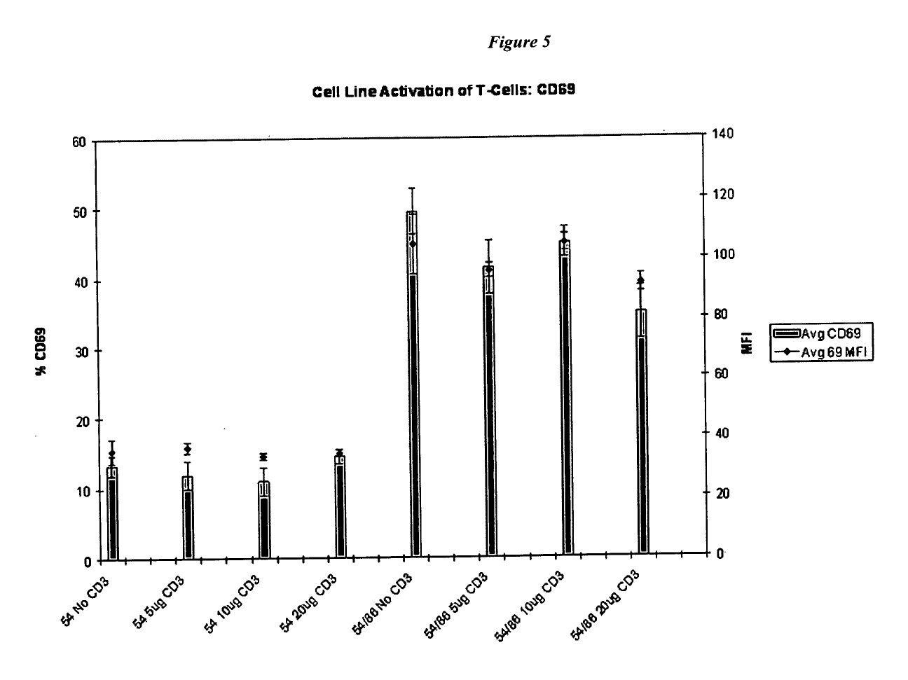

FIG. 6 shows the effect on transduction in primary CD4+ enriched T cells isolated from normal human apheresis product, after exposure to unmodified or modified lentiviral vectors expressing GFP as a marker gene. Cells were exposed to no stimulatory antibody (nada), soluble anti-CD3 antibody (CD3), or anti-CD3/28 antibody-coated beads (B). Each group of three conditions was then exposed to one of several vector groups at an MOI of 20 that included unmodified control vector (CV), or vector produced in a cell line expressing CD54 (54) or CD86 (86) or both (54/86). In addition to the level of GFP expression measured as the percentage of modified cells, also shown are the relative CD25 and CD69 protein levels. n=3 and standard error from the mean is shown.

FIG. 7 shows the effect on transduction in primary CD4+ enriched T cells isolated from normal human apheresis product, after exposure to unmodified or modified lentiviral vectors expressing GFP as a marker gene. Cells were exposed to no stimulatory antibody (nada), soluble anti-CD3 antibody (CD3), or anti-CD3/28 antibody-coated beads (B). Each group of three conditions was then exposed to one of several vector groups at an MOI of 20 that included unmodified control vector (CV), or vector produced with transiently expressed CD54 (54) or CD86 (86) or both (54/86). In addition to the level of GFP expression measured as the percentage of modified cells, also shown are the relative CD25 and CD69 protein levels. n=1.

### DETAILED DESCRIPTION OF MODES OF PRACTICING THE INVENTION

This invention provides for retroviral packaging cells and vector packaging systems comprising them. The cells and systems may be used to prepare packaged vector particles that may be used to transduce a variety of target cells.

As described herein, there are multiple modes of practicing the invention. Non-limiting modes include the types of cells used, the types of vectors packaged, the types of ligands expressed, the constructs and methods used to express ligands, and the types of cells targeted by packaged vectors. Of course, and as would be understood by the skilled person, a packaging cell comprises at least one heterologous nucleic acid molecule which expresses or is capable of expressing, one or more viral factors necessary for the packaging of a vector. The one or more factors function in trans to permit the packaging of a viral nucleic acid vector into a virion or viral particle.

Thus in one mode, the invention provides a recombinant retroviral packaging cell comprising a first nucleic acid molecule capable of expressing, in said packaging cell, at least one membrane associated non-viral ligand which binds a cell surface molecule of a target cell. In some embodiments, the cell produces no viral particles in the absence of a second nucleic acid molecule that expresses viral factors or expresses, or is, a vector sequence. In another mode, the invention provides a retroviral vector packaging cell that endogenously expresses at least one membrane associated ligand capable of binding to a cell surface molecule of a target cell or tissue.

The invention may be practiced with a variety of cells. Embodiments include mammalian cells although other types of eukaryotic cells that support packaging of retroviral and lentiviral vectors may also be used. Types of cells include suspension cells and cells such as, but not limited to, COS, TE671, HT1080, Mv-1-Lu, and a human 293 cell line. Derivatives of the listed cells or of naturally occurring cells may also be used.

In further embodiments, a packaging cell expresses one or more viral proteins necessary in trans for vector packaging. In some embodiments, a retroviral packaging cell comprises
i) a heterologous nucleic acid molecule capable of expressing a viral or non-viral ligand which binds a cell surface molecule of a target cell and which optionally functions to mediate binding or fusion to a cell membrane containing said viral ligand with said target cell and/or stimulate the cell cycle transition from Gₚ to G_{1b}; or

ii) a heterologous nucleic acid molecule which is capable of expressing CD226 (DNAM-1), the wild type herpes simplex virus (HSV)-1 envelope protein, or another viral envelope protein.

Where the retroviral packaging cell comprises ii), the target cell is a hematopoietic stem cell; CD34+, CD33+, CD14+, or expresses nectin 1 or nectin-like proteins; a T cell; a dendritic cell or a B cell in the germinal center of a lymph node; or a fibroblast. In other embodiments, a packaging cell of the invention is derived from a cell which interacts with the target cell or tissue in vivo.

Non-limiting examples of trans acting proteins include structural proteins, envelope (env) proteins, accessory proteins, proteins which facilitate fusions with target cell membranes, and the Gag and/or Pol proteins of any retrovirus or lentivirus, including, but not limited to, Moloney Murine Leukemia Virus, Rous Sarcoma Virus, RD114, BaEV, GALV, SSAV, FeLV-B, amphotropic murine leukemia viruses (MLV-A), human immunodeficiency virus, avian leukosis virus and NZB virus. The GAG and/or POL proteins may also be modified, synthetic, or chimeric GAG/POL constructs.

Examples of envelope proteins include, but are not limited to, an amphotropic envelope, an ecotropic envelope, or a xenotropic envelope, or may be an envelope including amphotropic and ecotropic portions. The protein also may be that of any of the above mentioned retroviruses and lentiviruses. Alternatively, the env proteins may be modified, synthetic or chimeric env constructs, or may be obtained from non-retroviruses, such as vesicular stomatitis virus and HVJ virus. Specific non-limiting examples include the envelope of Moloney Murine Leukemia Virus (MMLV), Rous Sarcoma Virus, and the feline endogenous virus RD114; gibbon ape leukemia virus (GALV) envelope; baboon endogenous virus (BaEV) envelope; simian sarcoma associated virus (SSAV) envelope; amphotropic murine leukemia virus (MLV-A) envelope; human immunodeficiency virus envelope; avian leukosis virus envelope; the endogenous xenotropic NZB viral envelopes; and envelopes of the paramyxoviridiae family such as, but not limited to the HVJ virus envelope.

The types of vectors packaged include those based upon any virus. Vectors derived from retroviruses, including avian reticuloendotheliosis virus (duck infectious anaemia virus, spleen necrosis virus, Twiehaus-strain reticuloendotheliosis virus, C-type retrovirus, reticuloendotheliosis virus Hungary-2 (REV-H-2)), and feline leukemia virus (FeLV)), are some non-limiting examples. Retroviral genomes have been modified for use as a vector (Cone & Mulligan, Proc. Natl. Acad. Sci., USA, 81:6349-6353, (1984)). Non-limiting examples of retroviruses, or members of the retroviridae family, which may be used as vectors of the invention include lentiviruses, such as human immunodeficiency viruses (HIV-1 and HIV-2), feline immunodeficiency virus (FIV), simian immunodeficiency virus (SIV), Maedi/Visna virus, caprine arthritis/encephalitis virus, equine infectious anaemia virus (EIAV), and bovine immunodeficiency virus (BIV); avian type C retroviruses, such as the avian leukosis virus (ALV); HTLV-BLV retroviruses, such as bovine leukaemia virus (BLV), human T-cell lymphotropic virus (HTLV), and simian T-cell lymphotropic virus; mammalian type B retroviruses, such as the mouse mammary tumor virus (MMTV); mammalian type C retroviruses, such as the murine leukaemia virus (MLV), feline sarcoma virus (FeSV), murine sarcoma virus, Gibbon ape leukemia virus, guinea pig type C virus, porcine type C virus, wooly monkey sarcoma virus, and viper retrovirus; spumavirus (foamy virus group), such as human spumavirus (HSRV), feline synctium-forming virus (FeSFV), human foamy virus, simian foamy virus, and bovine syncytial virus; and type D retroviruses, such as Mason-Pfizer monkey virus (MPMV), squirrel monkey retrovirus, and langur monkey virus.

The term “vector” or “plasmid” refers to a nucleic acid molecule capable of transporting a nucleic acid sequence between different cellular or genetic environments. Non-limiting vectors include those capable of autonomous replication and expression of nucleic acid sequences present therein. Vectors may also optionally comprise selectable markers that are compatible with the cellular system used. One type of vector for use in the practice of the invention is maintained as an episome, which is a nucleic acid capable of extra-chromosomal replication. Another type is a vector which is stably integrated into the genome of the cell in which it is introduced.

The vectors of the invention may include genetic material encoding a “payload” which is expressed in the packaging cell and/or the target cell after delivery of the vector to a target cell. The fact that the vectors are packaged into a particle also permits the “payload”, or a biological product that is produced upon expression of the genetic material encoding the “payload”, to be incorporated into the packaged particle for delivery to a target cell. One “payload” of the invention is a therapeutic agent that is encoded by the vector's genome. Of course a “payload” that is a polypeptide or a nucleic acid (such as RNA) may also be expressed in the packaging cell and physically present in the packaged particle in addition to, or instead of, being expressed in the target cell. In some embodiments, a “payload” is not toxic or is minimally toxic to the packaging cell used.

Non-limiting examples of genetic material encoding a therapeutic agent include polynucleotides encoding tumor necrosis factor (TNF) genes, such as TNF-α; genes encoding interferons such as Interferon-α, Interferon-β, and Interferon-γ; genes encoding interleukins such as IL-1, IL-1β, and Interleukins 2 through 14; genes encoding GM-CSF; genes encoding adenosine deaminase, or ADA; genes which encode cellular growth factors, such as lymphokines, which are growth factors for lymphocytes; genes encoding epidermal growth factor (EGF), and keratinocyte growth factor (KGF); genes encoding soluble CD4; Factor VIII; Factor IX; cytochrome b; glucocerebrosidase; T-cell receptors; the LDL receptor, ApoE, ApoC, ApoAI and other genes involved in cholesterol transport and metabolism; the alpha-1 antitrypsin (α1AT) gene; the insulin gene; the hypoxanthine phosphoribosyl transferase gene; the CFTR gene; negative selective markers or “suicide” genes, such as viral thymidine kinase genes, such as the Herpes Simplex Virus thymidine kinase gene, the cytomegalovirus virus thymidine kinase gene, and the varicella-zoster virus thymidine kinase gene; Fc receptors for antigen-binding domains of antibodies, antisense sequences which inhibit viral replication, such as antisense sequences which inhibit replication of hepatitis B or hepatitis non-A non-B virus; antisense c-myb oligonucleotides; and genes encoding antioxidants such as, but not limited to, manganese superoxide dismutase (Mn—SOD), catalase, copper-zinc-superoxide dismutase (CuZn—SOD), extracellular superoxide dismutase (EC-SOD), and glutathione reductase; the multidrug resistance (MDR) gene; polynucleotides encoding ribozymes; antisense polynucleotides; genes encoding secretory peptides which act as competitive inhibitors of angiotensin converting enzyme, of vascular smooth muscle calcium channels, or of adrenergic receptors, and polynucleotides encoding enzymes which break down amyloid plaques within the central nervous system.

Genetic material encoding the following may also be used: tissue plasminogen activator (tPA); urinary plasminogen activator (urokinase); hirudin; the phenylalanine hydroxylase gene; neuron nitric oxide synthase; endothelial nitric oxide synthase; vasoactive peptides; angiogenic peptides; anti-angiogenic peptides; the dopamine gene; the dystrophin gene; the β-globin gene; the α-globin gene; the HbA gene; protooncogenes such as the ras, src, and bcl genes; tumor-suppressor genes such as p53 and Rb; the LDL receptor; the heregulin-α protein gene, for treating breast, ovarian, gastric and endometrial cancers; and monoclonal antibodies specific to epitopes contained within the β-chain of a T-cell antigen receptor. It is to be understood, however, that the scope of the present invention is not to be limited to any particular therapeutic agent.

In some embodiments of the invention, the payload can be a gene encoding a clotting factor (e.g., Factor VIII or Factor IX) useful in the treatment of hemophilia, or the gene can encode one or more products having another therapeutic effect. Examples of suitable genes include those that encode cytokines such as TNF, interleukins (interleukins 1-12), interferons (α, β, and γ-interferons), T-cell receptor proteins and Fc receptors for binding to antibodies.

The vectors of the invention are useful in the treatment of a variety of diseases including but not limited to infectious diseases such as viral infections like HIV and herpes infections, genetic based disorders such as cancer, adenosine deaminase deficiency, sickle cell anemia, thalassemia, hemophilia, diabetes, α-antitrypsin deficiency, brain disorders such as Alzheimer's disease, and other illnesses such as growth disorders and heart diseases, for example, those caused by alterations in the way cholesterol is metabolized and defects of the immune system.

In other embodiments, the vectors of the invention may include a negative selectable marker, such as, for example, a viral thymidine kinase gene, such as the Herpes Simplex Virus thymidine kinase (TK) gene. Such vectors may be administered to cancer cells (in particular to tumor cells) in a human patient in vivo or used ex vivo. The vectors then transduce the tumor cells. After the vectors have transduced the tumor cells, the patient is given an interaction agent, such as gancyclovir or acyclovir, which interacts with the protein expressed by the negative selectable marker in order to kill all replicating cells (i.e., the cancer or tumor cells) which were transduced with the vector encoding the negative selectable marker.

The vectors of the invention are designed to bind and infect target cells of interest. In some embodiments, the target cell is an antigen presenting cell (APC), a cell of the hematopoietic lineage, a stem cell, a hematopoietic stem cell, a lymphocyte, a neuron, an endothelial cell, or a tumor cell. Where the target is a hematopoietic stem cell, the packaging cell is optionally a bone marrow stromal cell. Hematopoietic stem cell targets may be CD34+ and CD38−/CXCR4+, or CD34+ and CD38low/CXCR4+. Other stem cells include CD34+ cells isolated from fetal tissues such as bone marrow or liver, CD34+ precursor human embryonic stem cells (hESC), CD133+ stem cells of either hematopoietic origin, or other origin as described above, or cells manifesting the side population (SP) phenotype. The ligand is optionally SDF-1, VLA-4, VLA-5, or LFA-1. The invention also includes targeting CD34+ cells by use of integrin as the ligand.

The invention may be practiced with a variety of naturally occurring or engineered membrane associated ligands that bind a cell surface molecule of a target cell and facilitate viral particle transduction of target cells. Such a ligand may function to mediate fusion of a cell membrane containing said ligand with the cell membrane of a target cell. Embodiments include any ligand molecules known to have a role in cell-cell adhesion or to have an activating or stimulatory (inducing entry into or transition through all or part of the cell cycle or cell proliferation or cell growth) activity on target cells. Included are co-stimulatory molecules known to have a synergistic action in T cell proliferation while the CD3/TCR complex is engaged by a natural ligand or by a specific antibody (Ab). Thus a co-stimulatory molecule that binds a T cell surface molecule to activate T cell proliferation when the CD3/TCR complex of said T cell is bound by a natural or artificial ligand or by a specific Ab may be used in the practice of the invention. In some embodiments, packaging cells express at least two such ligands, such as, but not limited to, one that facilitates cell-cell adhesion and another that facilitates target cell stimulation. In other embodiments, the membrane associated non-viral ligand(s) is that of a hematopoietic cell or is a transmembrane protein(s).

Other non-limiting examples of membrane associated ligands are antibodies, including chimeric, humanized, monoclonal, single chain forms, and fragments thereof, that function as membrane associated ligands as described herein. Non-limiting examples include an antibody or antibody fragment that binds LFA-1, CD18, CD11b, CD11c, CD11d or CD43. Further non-limiting examples are microbial components that function as ligands as described herein. A microbial protein that binds LFA-1, CD18, CD11b, CD11c, CD11d or CD43 represents a non-limiting example.

Specific exemplary, and non-limiting examples of ligands include CD28; CD28BP (see Lazetic et al., J. Biol. Chem., 277(41):38660-38668 (2002)); B7-H₁, B7-H₂ (also known as ICOS-L, B7RP-1, GL50), B7-H₃, B7-H₄, and other related B7 molecules; PD-1 binding molecules such as PD-L₂ and PD-L₁; OX40 ligands (CD154) which bind OX40/CD134); 4-1BB ligands which binds 4-1BB/CD137; LFA-1 (which is a complex of CD11a with CD18) binding molecules, such as ICAM-1/CD54, ICAM-2/CD102, ICAM-3/CD50, ICAM-4/LW, or ICAM-5/telencephalin; antibodies or microbial molecules that bind LFA-1, CD11a, CD18, CD11b, CD11c, CD11d, or CD43; ligands for CD2 (LFA-2), such as CD15, CD48, CD58, or CD59; ligands for CD5 such as CD72; and ligands for CD58 (LFA-3), such as CD2. Of course any combination of such ligands may also be used in the practice of the invention.

Other exemplary and non-limiting examples include ligands that bind the surface of target T cells as well as other cells, including B cells (e.g. via CD40), CD34 expressing hematopoietic progenitors (e.g. via CD62L), and other cells susceptible to retroviral and/or lentiviral infection/replication. Additional examples of cell surface molecules for use as a ligand include LFA-3 (also called CD58); FasL (Fas ligand); CD70; B7-H1 (also called PD-L1); B7-H2; B7-H3 (also called B7RP-2); B7-H4; CD2; CD3 or CD3/TCR complex; CD11a; CD26; CD27; CD28; CD30L; CD32; CD38; CD40L (also called CD154); CD45; CD49; CD50 (also called ICAM-3); CD54 (also called ICAM-1); CD80 (also called B7.1); CD86 (also called B7.2); CD100; CD122; CD137L (also called 4-1BB Ligand); CD153; CTLA-4 (also called CD152); ICOS; OX40L (also called CD134); PD-1; PD-L2 (also called B7-DC); SLAM (also called CD150); TIM-1; TIM-2; TIM-3; TIM-4; and 2B4 (also called CD244). Some ligands may be optionally co-expressed with MHC class II. Of course any combination of such ligands may also be used in the practice of the invention.

Nucleic acids encoding ligand or viral factors for use in the instant invention may be obtained or prepared by any suitable or convenient method known, including PCR amplification of known sequences.

One type of ligand molecule is that of stimulatory or costimulatory molecules such as B7-1 (previously known as B7 and also known as CD80) and B7-2 (also known as CD86) which binds to CD28. B7-1 and B7-2 are related homodimeric glycoproteins that are part of the immunoglobulin superfamily. Other stimulatory or costimulatory molecules are anti-CD28 antibodies or functional portions thereof, including F_{ab} portions that bind CD28. In some embodiments, the packaging cell expresses CD86, but not CD54, as a ligand.

Another type of ligand is a cell-cell adhesion molecule. These include the various ICAM molecules, which include intercellular adhesion molecule (ICAM) ICAM-1, ICAM-2, ICAM-3, lymphocyte function-associated antigen (LFA) LFA-1 and LFA-3. The ICAM-related members all bind to the T cell integrin, LFA-1. In addition to being expressed on APCs including dendritic cells, macrophages and B cells, ICAM-1 and ICAM-2 are also expressed on endothelium, thereby mediating cell adhesion and possible transduction with packaged vectors of the instant invention. ICAM-3 is only expressed on leukocytes and so may be targeted for binding with a ligand of the instant invention. In some embodiments, the packaging cell expresses CD86, but not ICAM-1 (CD54), ICAM-2, or ICAM-3, as a ligand.

The interaction between ICAM-1, -2 and -3 has been observed to be synergistic with a second binding interaction between LFA-3 (CD58) and LFA-2 (CD2) with LFA-1 (CD11a and CD18). These ICAM molecules may thus be co-expressed with either CD58, CD11a and CD18, or CD2 to permit targeting of cells with surface molecules that bind CD58, CD11a and CD18, or CD2. In some embodiments, LFA-1, LFA-2, or LFA-3 is not co-expressed with CD86.

The invention may also be practiced with the use of survival molecules as the ligand. Survival molecules are defined as those that play a role in cellular responses ranging from stimulation to induction of cell death. A survival molecule may be a protein that is exposed on the surface of a cell or other cell surface macromolecule such as a carbohydrate or lipid. Such cell surface molecules are also referred to as receptors on the cell surface. Survival molecules for use in the practice of the instant invention include the Fas ligand.

Other molecules include TNF-receptor, TNF, and CD70, a Type II transmembrane protein that is a member of the TNF family that binds to CD27. CD27 is expressed on resting T and B cells while CD70 is expressed on activated T and B cells. Binding of CD70 to its receptor, CD27, induces T-cell costimulation and the interaction may be important for the recruitment of T cells from the unprimed T cell pool. Under other certain conditions, activation of the TNF receptor by TNF results in a similar response.

Other non-limiting cell surface binding molecules are antibodies or ligands for the FLT-3 ligand, TPO, and Kit ligand receptors, which make cells expressing the receptors, such as hematopoietic stem cells, more receptive to vector transduction. Additional non-limiting cell surface binding molecules are antibodies or ligands for GM-CSF and IL-4 receptors, which make dendritic cells or their precursors, such as monocytes, CD34 positive stem cells, or their differentiated progenitor cells on the dendritic cell lineage, more receptive to vector transduction. Other cell surface binding molecules include molecules found on cell surfaces which bind the surface of another cell.

Additional examples of cell surface binding molecules include polypeptides, nucleic acids, carbohydrates, lipids, and ions, all optionally complexed with other substances. In some embodiments, the molecules bind factors found on the surfaces of blood cells, such as CD1a, CD1b, CD1c, CD1d, CD1e, CD2 or CD2R, CD3γ, CD3δ, CD3δ, CD4, CD5, CD6, CD7, CD8α, CD8β, CD9, CD10, CD11a, CD11b, CD11c, CDw12, CD13, CD14, CD15, CD15u, CD16a, CD16b, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD44R, CD45 or CD45R or CD45RO or CD45RA or CD45RB or CD45RC, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD60b, CD60c, CD61, CD62E, CD62L, CD62P, CD63, CD64, CD65, CD65s, CD66a, CD66b, CD66c, CD66d, CD66e, CD66f, CD68, CD69, CD70, CD71, CD72, CD73, CD74, CD75, CD75s, CD77, CD79a, CD79b, CD80, CD81, CD82, CD83, CD84, CD85, CD86, CD87, CD88, CD89, CD90, CD91, CDw92, CDw93, CD94, CD95, CD96, CD97, CD98, CD99, CD100, CD101, CD102, CD103, CD104, CD105, CD106, CD107a, CD107b, CD108, CD109, CD110, CD111, CD112, CD113, CD114, CD115, CD116, CD117, CD118, CDw119, CD120a, CD120b, CD121a, CDw121b, CD122, CD123, CD124, CDw125, CD126, CD127, CDw128a, CDw128b, CD129, CD130, CD131, CD132, CD133, CD134, CD135, CDw136, CDw137, CD138, CD139, CD140a, CD140b, CD141, CD142, CD143, CD144, CDw145, CD146, CD147, CD148, CDw149, CD150, CD151, CD152, CD153, CD154, CD155, CD156a, CD156b, CD156c, CD157, CD158, CD159a, CD159b, CD160, CD161, CD162, CD162R, CD163, CD164, CD165, CD166, TCRζ, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CDw186, CD191, CD192, CD193, CD195, CD196, CD197 or CDw197, CDw198, CDw199, CD200, CD201, CD202b, CD203c, CD204, CD205, CD206, CD207, CD208, CD209, CDw210, CD212, CD213a1, CD213a2, CDw217, CDw218a, CDw218b, CD220, CD221, CD222, CD223, CD224, CD225, CD226, CD227, CD228, CD229, CD230, CD231, CD232, CD233, CD234, CD235a, CD235b, CD235ab, CD236, CD236R, CD238, CD239, CD240CE, CD240D, CD240DCE, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD271, CD272, CD273, CD274, CD275, CD276, CD277, CD278, CD279, CD280, CD281, CD282, CD283, CD284, CD289, CD292, CDw293, CD294, CD295, CD296, CD297, CD298, CD299, CD300a, CD300c, CD300e, CD301, CD302, CD303, CD304, CD305, CD306, CD307, CD309, CD312, CD314, CD315, CD316, CD317, CD318, CD319, CD320, CD321, CD322, CD324, CDw325, CD326, CDw327, CDw328, CDw329, CD331, CD332, CD333, CD334, CD335, CD336, CD337, CDw338, and CD339. Small letters (e.g. “a” or “b”) indicate complex CD molecules composed of multiple gene products or belonging to families of structurally related proteins. The notation “w” refers to putative CD molecules that have not yet been fully confirmed. A more complete listing of CD molecules is found via http://at mpr.nci.nih.gov/prow/. Of course any combination of such ligands may also be used in the practice of the invention.

Other non-limiting examples are molecules that bind factors found on the surfaces of lymphocytes, T cells and leukocytes, such as CD2, CD3γ, CD3δ, CD3ε, CD5, CD6, CD7, CD8α, CD8β, CD9, CD11a, CD18, CD25, CD26, CD27, CD28, CD29, CD30, CD37, CD38, CD39, CD43, CD44, CD45R, CD46, CD48, CD49a, CD49b, CD49c, CD49d, CD49e, CD49f, CD50, CD53, CD54, CD56, CD57, CD58, CD59, CDw60, CD62L, CD68, CD69, CDw70, CD71, CD73, CDw75, CDw76, CD84, CD85, CD86, CD87, CD89, CD90, CD94, CD96, CD97, CD98, CD99, CD100, CD101, CD103, CD107a, CD107b, CDw108, CDw109, CD118, CD119, CD120b, CD121a, CD122, CDw124, CDw127, CDw128a, CDw130, CD132, CD134, CDw137, CD140a, CD140b, CD143, CD146, CD148, CD152, CD153, CD154, CD155, CD161, CD162, CD165, CD166, and TCRζ. Of course any combination of such ligands may also be used in the practice of the invention.

A variety of constructs and methods may be used to express viral factors and ligands of the invention. Non-limiting constructs include those that comprise a suitable promoter for regulating expression of the ligand in the packaging cell used. These include inducible and constitutive promoters that are operably linked to nucleic acid sequences encoding viral factors and ligands as described herein.

The invention may be practiced with the use of plasmid expression vectors in substantially pure form capable of expressing ligands and viral factors as described herein. Sequences encoding the ligands and viral factors are thus used to produce the corresponding recombinant proteins. Helper vectors capable of directing the expression of such sequences may also be referred to herein as “expression vectors” or “expression plasmids”.

Expression vectors contain a promoter sequence in the regulatory region which facilitates the transcription of (is operably linked to) an inserted nucleic acid sequence in the host. The sequence may encode a ligand or a “payload” of the invention. The vector may contain an inducible promoter sequence to limit selection pressure against cells that constitutively express a sequence. The expression vector also typically contains an origin of replication as well as specific genes which allow phenotypic selection of the transformed cells. Operatively linked refers to the covalent linkage of nucleic acid sequences to result in the joining of functional elements such as response elements in regulatory regions with promoters and coding sequences as described herein.

Vectors for use in the instant invention may be those compatible with eukaryotic cells. Eukaryotic cell expression vectors are known in the art and contain promoters for expression of sequences encoding ligands and viral factors of the invention. These expression vectors also contain distinct sequence elements that are required for accurate and efficient polyadenylation as well as optional splicing signals for generating mature mRNA. They may also optionally contain viral replicons to increase the level of expression of encoded genes. High level expression may also be achieved using inducible promoters, including, but not limited to, the metallothionine IIA promoter and heat shock promoters.

The invention also provides a method of producing a recombinant retroviral packaging cell. The method comprises introducing, into a cell, 1) a nucleic acid molecule capable of expressing, in said packaging cell, a viral gene product necessary for packaging and/or replication of a retrovirus, and 2) at least one nucleic acid molecule capable of expressing, in said packaging cell, at least one membrane associated non-viral ligand(s) which bind a cell surface molecule of a target cell. An additional method comprises introducing, into a cell, 1) a nucleic acid molecule capable of expressing, in said packaging cell, a viral gene product necessary for packaging and/or replication of a retrovirus, and 2) at least one nucleic acid molecule capable of increasing the expression of, in said packaging cell, at least one membrane associated non-viral ligand(s) which bind a cell surface molecule of a target cell.

In some embodiments, the invention further provides for stable transfection to provide long-term, high-yield production of recombinant ligands and viral factors. Stable transfection refers to the integration of genetic material into a cellular genome such that the material is not lost over the course of subsequent cycles of cell replication and division. Alternatively, cells can be transiently transfected such that the genetic material is not stable and may be lost from the cell during subsequent cycles of cell replication and division.

Packaging cells can be transformed with a cDNA controlled by appropriate expression control elements (e.g., promoter and enhancer sequences, transcription terminators, polyadenylation sites, etc.), and an optional selectable marker. The selectable marker confers resistance to the selection and allows cells to stably integrate the plasmid into their chromosomes and grow to form clones that can be expanded into cell lines.

The invention also contemplates the presence of more than one ligand and/or viral factor encoding sequence being present within the same vector and under the control of one or separate (optionally multiple) regulatory elements such as promoters. Thus, all possible combinations of helper vector construction for producing the recombinant ligand and/or viral factor proteins of this invention in a packaging cell are contemplated.

The types of cells targeted by packaged vectors (or target cells) include, but are not limited to, primary cells, including blood cells, which include all forms of nucleated blood cells as well as progenitors and precursors thereof; liver cells; endothelial cells; lymphocytes; and tumor cells, including malignant and non-malignant tumor cells. For administration to such cells, the packaged vectors may be administered to an animal, such as a human, via ex vivo or in vivo techniques.

Additionally, the invention provides a method of producing or packaging a viral vector. The method comprises 1) introducing, into a packaging cell of the invention, a nucleic acid molecule encoding or comprising a retroviral vector, and 2) culturing said cell under conditions wherein said cell packages said vector into a particle comprising one or more membrane associated non-viral ligand(s) of the packaging cell. The ligand(s) are as described herein, and so may bind a cell surface molecule of a target cell. In some embodiments, the nucleic acid molecule encoding or comprising a retroviral vector is the “second” nucleic acid introduced into the packaging cell.

Having now generally described the invention, the same will be more readily understood through reference to the following examples which are provided by way of illustration, and are not intended to be limiting of the present invention, unless specified.

### EXAMPLES

### Example 1

### Lentivirus Vector Membrane-Incorporated CD54-Mediated Enhancement of Transduction

VRX494 is a minimal HIV-based lentivirus vector containing the cppt/CTS sequence, a 937-base antisense payload targeted to the HIV envelope gene, and a GFP-marker gene which may be removed in further embodiments. The latter two nucleic acid sequences are both expressed under the control of the LTR promoter. VRX494 was produced by transient transfection in 293 FT cells in the presence of a single helper plasmid (thus a 293 based packaging cell) according to published procedures (Lu et al., 2004) to produce a G protein pseudotyped viral particle. To examine if expression of CD54 on the cell surface of packaging cells would result in expression on the vector membrane envelope and thus enhance transduction, a CD54-expressing construct (VRX588) was made. VRX494 was produced either in the presence or absence of VRX588 resulting in vector containing or not containing CD54 at the viral particle envelope membrane surface. Vector-containing supernatants were collected at various intervals, pooled and centrifuged. Vector pellets were reconstituted in buffer, and then titrated on HeLa-tat cells according to published procedures (Lu et al., 2004). CD4+ T lymphocytes were isolated from human PBMCs by MACS positive selection. Purified cells were cultured in T cell medium (Xvivo 15 complemented with 10 mM NAC, 5% human serum, 100 units/ml IL-2, and gentamycin) overnight at a concentration of 1×10⁶ per well in the presence of CD3/CD28 stimulatory beads at a ratio of 3 beads per cell, which may affect CD11a/CD18 (LFA-1) conformation to make it more susceptible to binding by CD54 (ICAM-1) on the vector particles. Transduction was performed in triplicate at an MOI of 20 in the presence or absence of retronectin. Cultures were incubated in the presence of vector for 3 days at 37° C. in 5% CO₂. Unused vector was washed away, and cells were subsequently re-plated at 0.5×10⁶ cells per ml. Cells were maintained at this concentration up to day 7 by expanding the medium culture volume into appropriate containers. On day 7, cultures were collected, washed twice in PBS, counted, and dry pelleted at 10⁶ cells/vial for determination of the average vector copy number per cell using TaqMan PCR according to SOP.

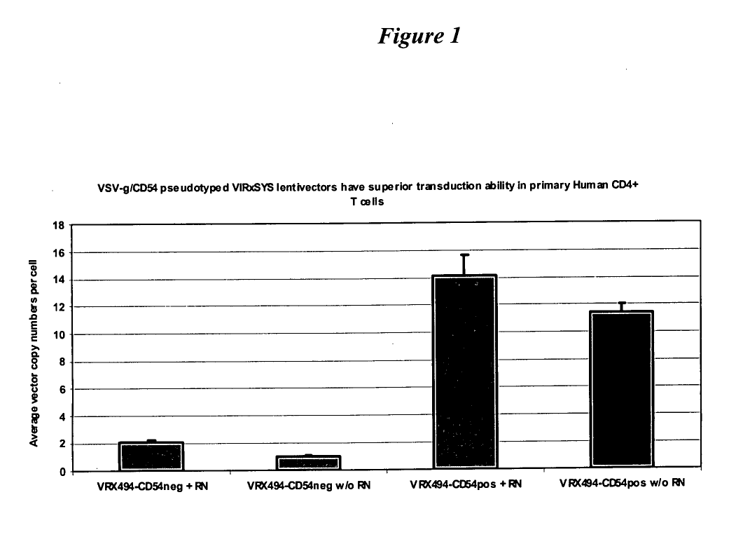

In the presence of retronectin, which is a compound known for its ability to enhance transduction, the presence of CD54 increased copy numbers in target primary CD4 T lymphocytes approximately 7-fold when compared to CD54 minus controls. In the absence of retronectin, CD54 enhanced target copy numbers 10-fold over CD54 minus controls. Similar levels of transduction were achieved between CD54 positive vector-transduced cells in the presence or absence of retronectin (FIG. 1). This indicates that retronectin has a relatively minor positive effect on transduction while the presence of CD54 has a major effect.

### Example 2

### Enhanced Cell Stimulation and Expansion After Transduction with Lentiviral Vectors Packaged with CD86 Expressed on the Vector Membrane

293F cells were modified to contain CD54, CD86, or both proteins by transfection of cells with expression constructs encoding the proteins. Transfected cells were tested at several points by flow cytometric evaluation to ensure expression of the proper proteins on the cell surface. Cells were stained with anti-CD54-PE or anti-CD86-PE, and propidium iodide for viability. Isotype controls anti-mouse IgG1-PE and anti-Mouse IgG2a were used to detect baseline staining.

For evaluation of stimulation and expansion, frozen CD4+ T cells, isolated from a human apheresis product that had subsequently been purified for CD4 T cells by CD4+ enrichment (using a Miltenyi apparatus) were cultured in X-vivo 15 medium containing 5% human AB serum, 50 μg/ml gentamycin, 100 U/ml IL-2 and 1.6 mg/mL N-acetyl cysteine (NAC). Cells were seeded at a concentration of 1×10⁶ cells/ml in one of three conditions: no additions, anti-CD3 antibody (clone OKT-3) at a concentration of 5 mg/ml, or with anti-CD3/CD28 microbeads at a concentration of 3 beads per cell. For each of these three conditions, no vector, control vector, or control vector containing in their membrane CD54, CD86, or both proteins together, were tested.

On days 1 and 2, cells in all wells were gently resuspended to break up any clumping. On day 3, cells were washed twice, and counted and sized using the Z2 Coulter Counter. On day 7, cells were counted and sized again, and analyzed for expression of green fluorescent protein (GFP), which is a marker gene expressed by the lentiviral vectors used. Also tested at this time were markers of activation, CD25 and CD69 expression using the an anti-CD25-PE, or anti-CD69-PE and propidium iodine, with an IgGK1-PE isotype control for baseline staining.

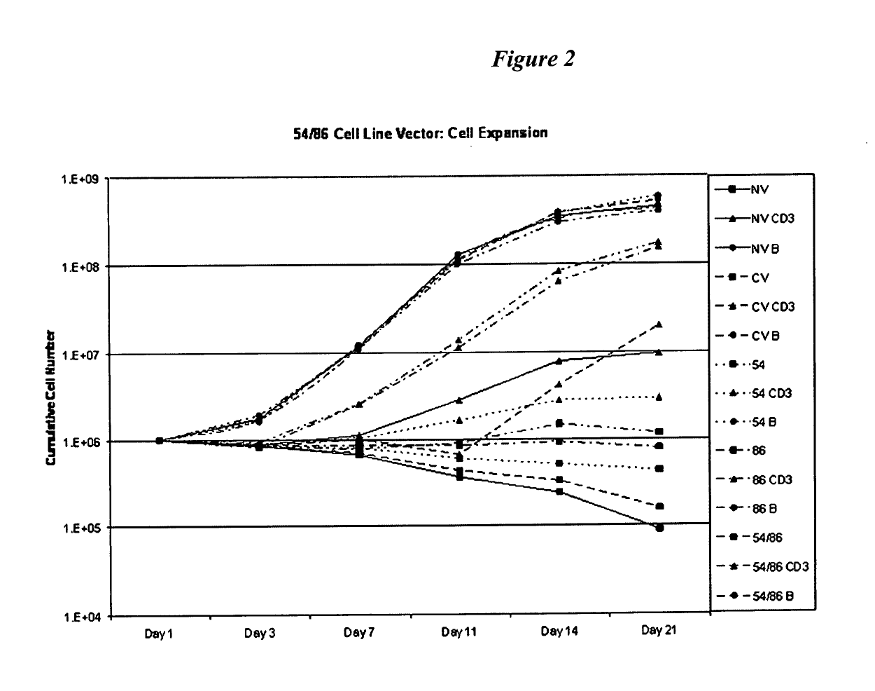

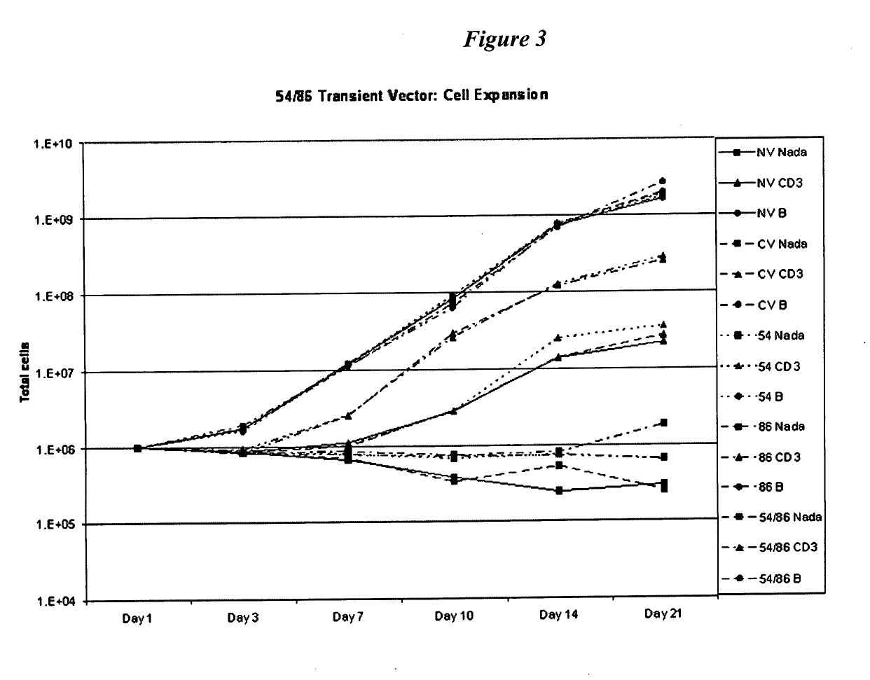

Two sets of experiments were performed. The first used vector produced in the 293 cell lines described above. The second used vector that was produced in cells using a simple transient cotransfection technique, without the prior establishment of cell lines. The expansion results from these experiments are shown in FIGS. 2 and 3, respectively. Cells treated with CD86-surface-expressing lentiviral vectors in combination with soluble anti-CD3 antibody, demonstrated significantly enhanced growth over cells treated with normal (unmodified) vectors. CD54 did not appear to play a role in enhancing or retarding cell growth. There was no significant difference observed between cells treated with vector produced using the 293F cell lines, or via transient transfection.

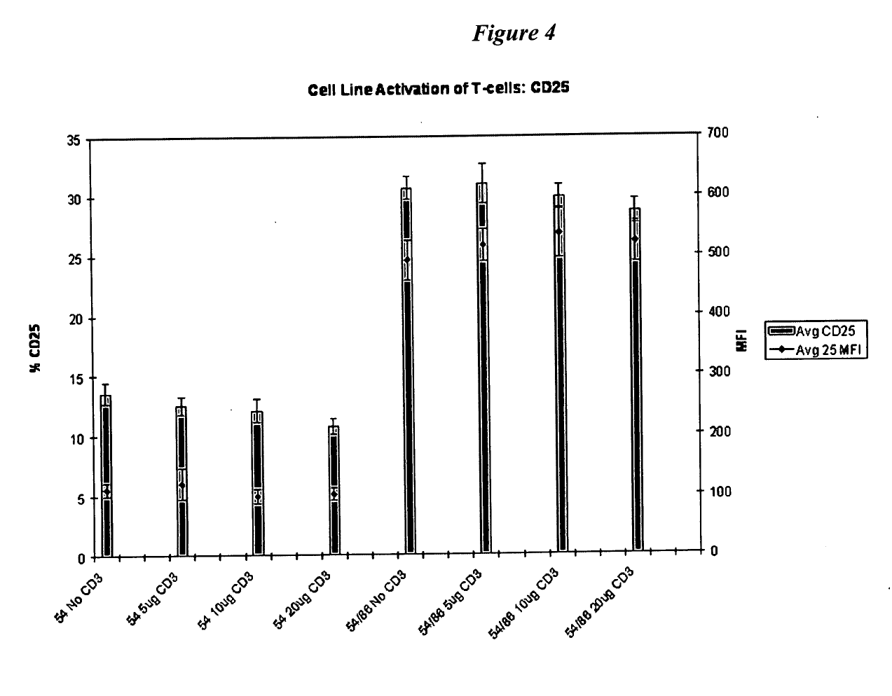

Co-culture on 293 cells expressing CD86 and CD54 enhanced the expression of activation markers CD25 and CD69 on CD4+ T cells, compared to 293 cells expressing only CD54 (shown in FIGS. 4 & 5). These results support the finding that improved expansion in cells exposed to CD86-expressing cells are a result of cell activation.

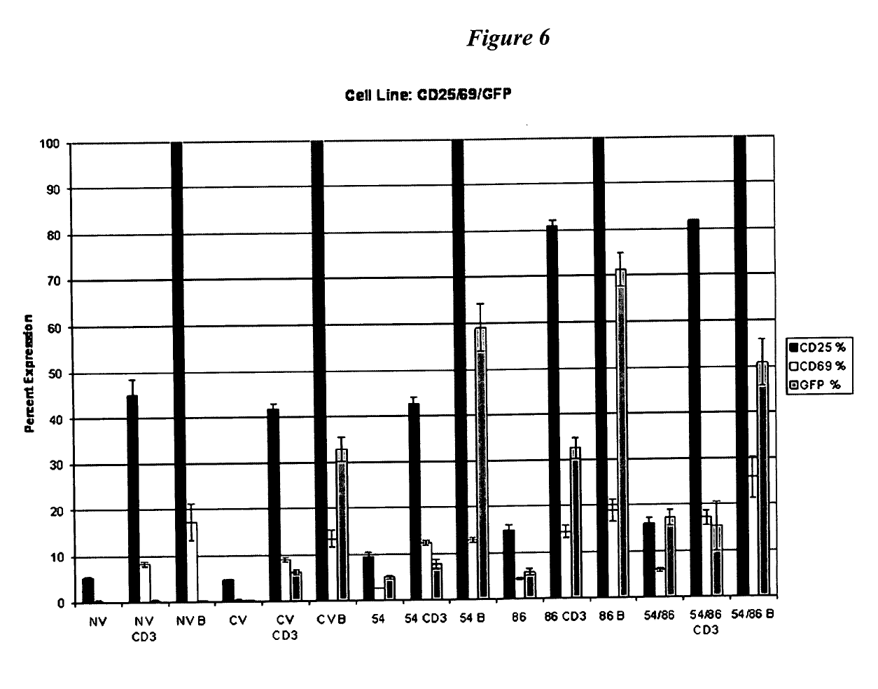

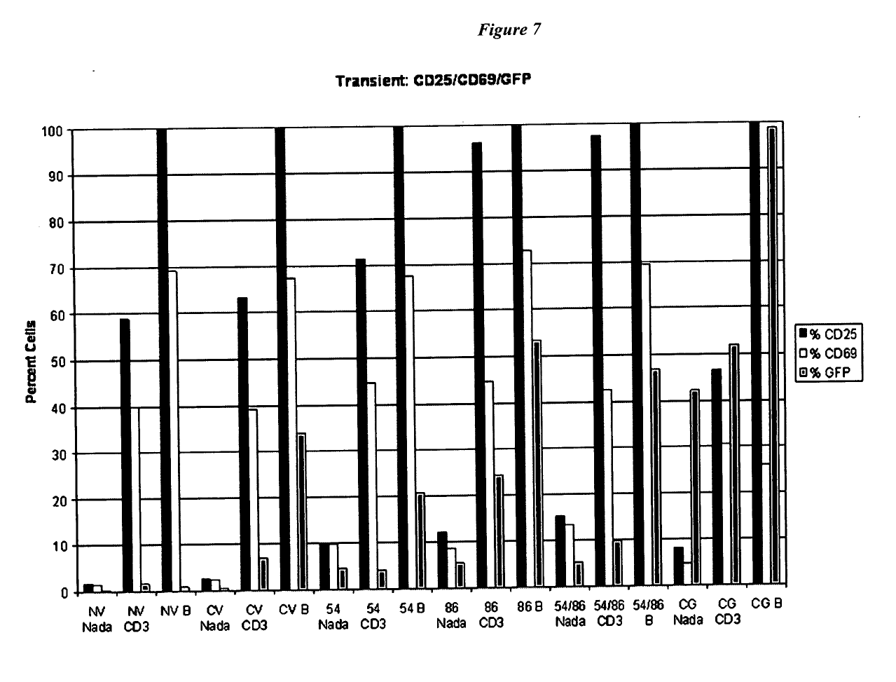

Finally, expression of CD86 on the surface of vector enhanced the level of GFP expression at day 7 for vectors produced by a 293F cell line or by transient transfection as described above (FIGS. 6 and 7). Although CD54 in combination with CD86 has previously been shown to improve the number of copies per cell of vector insertions, in this experiment, CD54 did not appear to contribute to the overall percentage of cells transduced with vector when compared to CD86 alone. The presence of CD86 alone increased the percentage of transduction from less than 10% to about 30%.

Together these data convincingly show that the incorporation of stimulatory ligands in the surface of vector particles indeed is capable of enhancing cell stimulation and growth.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

### REFERENCES

1. Barbeau B., Fortin J-F., Genois N., and Tremblay M. J. Modulation of human immunodeficiency virus type 1-induced syncitium formation by the conformational state of LFA-1 determined by a new luciferase-based syncytium quantitative assay. J. Virol. 72, 7125-7136 (1998).
2. Cavalieri S. et al. Human T lymphocytes transduced by lentiviral vectors in the absence of TCR-activation maintain an intact immune competence. Blood 102, 497-505 (2003).
3. Cone R. D., and Mulligan R. C. High-efficiency gene transfer into mammalian cells: generation of helper-free recombinant retrovirus with broad mammalian host range. Proc. Natl. Acad. Sci. 81, 6349-6353 (1984).
4. Costello E., Munoz M., Buetti E., Meylan P. R. A., Diggelmann H. & Thali M. Gene transfer into stimulated and unstimulated T lymphocytes by HIV-1-derived lentiviral vectors. Gene Ther. 7, 596-604 (2000).
5. Endres M. J., Jaffer S., Haggarty B., Turner J. D., Doranz B. J., O'Brien P. J., Kolson D. L., & Hoxie J. A. Targeting of HIV- and SIV-infected cells by CD4-chemokine receptor pseudotypes. Science 278:1462-1464 (1997).
6. Follenzi A., Ailles L. E., Bakovic S., Genua N. & Naldini L. Gene transfer by lentiviral vectors is limited by nuclear translocation and rescued by HIV-1 pol sequences. Nat. Gen. 25, 217-222 (2000).
7. Giguere J. F., Paquette J. S., Bounou S., Cantin R. & Tremblay M. J. New insights into the functionality of a virion-anchored host cell membrane protein: CD28 versus HIV type 1. J. Immunol. 169, 2762-2771 (2002).
8. Humeau L., et al. Efficient lentiviral vector-mediated control of HIV-1 replication in lymphocytes from diverse HIV infected patients grouped according to CD4 count and viral load. Mol. Ther. in press (2004).
9. Levine, B. L., Bernstein, W. B., Connors, M., Craighead, N., Lindsten, T., Thompson, C. B., and June, C. H. Effects of CD28 costimulation on long-term proliferation of CD4+ T cells in the absence of exogenous feeder cells. J. Immunol. 159: 5921-5930. (1997).
10. Levine, B. L., Bernstein, W. B., Aronson, N. E., Schlienger, K., Cotte, J., Perfetto, S., Humphries, M. J., Ratto-Kim, S., Birx, D. L., Steffens, C., Landay, A., Carroll, R. G., and June, C. H. Adoptive transfer of costimulated CD4+ T cells induces expansion of peripheral T cells and decreased CCR5 expression in HIV infection. Nat. Med. 8: 47-53. (2002).
11. Lu X., et al. Safe two-plasmid production for the first clinical lentivirus vector that achieves >99% transduction in primary cells using a one-step protocol. J. Gene Med. 6, 963-973 (2004).
12. Manganini M. et al. A human immunodeficiency virus type 1 pol gene-derived sequence (cPPT/CTS) increases the efficiency of transduction of human nondividing monocytes and T lymphocytes by lentiviral vectors. Hum. Gene Ther. 13, 1793-1807 (2002).
13. Maurice M., Verhoeyen E., Salmon P., Trono D., Russell S. J. & Cosset F. L. Efficient gene transfer into human primary blood lymphocytes by surface-engineered lentiviral vectors that display a T cell-activating peptide. Blood. 99, 2342-2350 (2002).
14. Mitsuyasu R. T. et al. Prolonged survival and tissue trafficking following adoptive transfer of CD4ζ gene-modified autologous CD4⁺ and CD8⁺ T cells in human immunodeficiency virus-infected subjects. Blood. 96, 785-792 (2000).
15. Naldini, L., et al. In Vivo gene delivery and stable transduction of nondividing cells by a lentiviral vector. Science 272, 263-267 (1996).
16. Naldini L. et al. Efficient transfer, integration, and sustained long-term expression of the transgene in adult rat brains injected with a lentiviral vector. Proc. Natl. Acad. Sci. 93, 11382-11388 (1996).
17. Paquette J. S., Fortin J. F., Blanchard L. & Tremblay M. J. Level of ICAM-1 surface expression on virus producer cells influences both the amount of virion-bound host ICAM-1 and human immunodeficiency virus type 1 infectivity. J. Virol. 72, 9329-9336 (1998).
18. Park F., Ohashi K., Chui W., Naldini L., and Kay M. A. Efficient lentiviral transduction of liver requires cell cycling in vivo. Nat. Gene. 24, 49-52 (2000).
19. Ranga, U. et al. Enhanced T cell engraftment after retroviral delivery of an antiviral gene in HIV-infected individuals. Proc. Natl. Acad. Sci. USA. 95, 1201-1206 (1998).
20. Schroers R., Davis C. M., Wagner H-J. & Chen S-Y. Lentiviral transduction of human T-lymphocytes with a RANTES intrakine inhibits human immunodeficiency virus type 1 infection. Gene Ther. 9, 889-897 (2002).
21. Verhoeyen E., Dardalhon V., Ducrey-Rundquist O, Trono D., Taylor N. & Cosset F. L. IL-7 surface-engineered lentiviral vectors promote survival and efficient gene transfer in resting primary T lymphocytes. Blood. 101, 2167-2174 (2003).

What is claimed is:

1. A recombinant retroviral packaging cell comprising a first nucleic acid molecule capable of expressing, in said packaging cell, at least one membrane associated non-viral ligand which binds a cell surface molecule of a target cell,
wherein said cell produces no viral particles in the absence of a second nucleic acid molecule.

2. The cell according to claim 1 wherein said retroviral packaging cell comprises
i) a heterologous nucleic acid molecule capable of expressing a viral ligand which binds a cell surface molecule of a target cell and which optionally functions to mediate fusion of a cell membrane containing said viral ligand with said target cell; or
ii) a heterologous nucleic acid molecule which is capable of expressing CD49d, CD54, CD80, CD86, or a combination thereof.

3. The cell according to claim 1 wherein said target cell is an antigen presenting cell (APC), a cell of the hematopoietic lineage, a stem cell, a hematopoietic stem cell, a lymphocyte, a neuron, an endothelial cell, or a tumor cell.

4. The cell according to claim 1 wherein said at least one membrane associated non-viral ligand(s) is
i) a co-stimulatory molecule that binds a T cell surface molecule to activate T cell proliferation when the CD3/TCR complex of said T cell is bound by a natural or artificial ligand or by a specific Ab, and/or
ii) a molecule that plays a role in cell-cell adhesion via binding to said cell surface molecule.

5. The cell according to claim 1 wherein
i) said at least one membrane associated non-viral ligand(s) activates said target cell to proliferate after binding said cell surface molecule;
ii) said at least one membrane associated non-viral ligand(s) comprise two such ligands;
iii) said membrane associated non-viral ligand(s) is that of a hematopoietic cell;
iv) said membrane associated non-viral ligand(s) are transmembrane protein(s); or
v) said membrane associated non-viral ligand(s) is CD28 or CD28BP or CD40 or CD62L or CD80 (B7-1) or CD86 (B7-2) or Fas ligand (FasL) or CD70 or LFA-3 (CD58) or B7-H1 (PD-L1) or B7-H2 or B7-H3 (B7RP-2) or B7-H4 or CD2 or CD3 or CD3/TCR complex or CD11a or CD26 or CD27 or CD28 or CD30L or CD32 or CD38 or CD40L (CD154) or CD45 or CD49 or CD50 (ICAM-3) or CD54 (ICAM-1) or CD100 or CD122 or CD137L (4-1BB Ligand) or CD153 or CTLA-4 (CD152) or ICOS or OX40L (CD134) or PD-1 or PD-L2 (B7-DC) or SLAM (CD150) or TIM-1 or TIM-2 or TIM-3 or TIM-4 or 2B4 (CD244), or a combination thereof.

6. The cell according to claim 1 wherein
i) said membrane associated non-viral ligand(s) is a B7 related molecule such as B7-H₁, B7-H₂ (also known as ICOS-L, B7RP-1, and GL50), B7-H₃, and B7-H₄;
ii) said membrane associated non-viral ligand(s) binds LFA-1, or a complex of CD11a with CD18, such as ICAM-2 (CD102), ICAM-3 (CD50), ICAM-4 (LW), or ICAM-5 (telencephalin); or
iii) said membrane associated non-viral ligand(s) is a PD-1 ligand, such as PD-L₂ or PD-L₁; an OX40 ligand (CD154) which binds OX40 (CD134); a 4-1BB ligand which binds 4-1BB (CD137); a ligand which binds LFA-2 (CD2), such as CD15, CD48, CD58, or CD59; a ligand which binds CD5, such as CD72; or a ligand which binds LFA-3 (CD58), such as CD2;
iv) said membrane associated non-viral ligand(s) is an antibody or antibody fragment that binds LFA-1, CD18, CD11b, CD11c, CD11d or CD43; or
v) said membrane associated non-viral ligand(s) is a microbial protein that binds LFA-1, CD18, CD11b, CD11c, CD11d or CD43.

7. The cell according to claim 2, wherein said retroviral packaging cell comprises a heterologous nucleic acid molecule which is capable of expressing the polio virus receptor CD155, the wild type herpes simplex virus (HSV)-1 envelope protein, or another viral envelope protein; and
i) the target cell is a hematopoietic stem cell;
ii) the target cell is CD34+, CD33+, CD14+, or expresses nectin 1;
iii) the target cell is a T cell;
iv) the target cell is a dendritic cell or a B cell in the germinal center of a lymph node;
v) the target cell is a fibroblast.

8. A method of producing a recombinant retroviral packaging cell according to claim 1, said method comprising
introducing, into a cell, a nucleic acid molecule capable of expressing, in said packaging cell, a viral gene product necessary for packaging and/or replication of a retrovirus, and
at least one nucleic acid molecule capable of expressing, in said packaging cell, at least one membrane associated non-viral ligand(s) which bind a cell surface molecule of a target cell.

9. A method of packaging a viral vector, said method comprising
introducing a second nucleic acid molecule encoding or comprising a retroviral vector into a recombinant cell according to claim 1, and
culturing said cell under conditions wherein said cell packages said vector into a particle comprising said membrane associated non-viral ligand(s) which bind a cell surface molecule of a target cell.

10. A retroviral vector packaging cell comprising a cell that endogenously expresses at least one membrane associated ligand capable of binding to a cell surface molecule of a target cell or tissue.

11. The packaging cell of claim 10 wherein the cell interacts with the target cell or tissue in vivo.

12. The packaging cell of claim 11 wherein the cell is a bone marrow stromal cell and the target cell is a hematopoietic stem cell.

13. The packaging cell of claim 10 wherein the ligand is an integrin and the target cells are CD34+ cells.

14. The packaging cell of claim 13 wherein the ligand is SDF-1, VLA-4, VLA-5, or LFA-1, and the hematopoietic stem cell is CD34+ and CD38−/CXCR4+, or CD34+ and CD38 low/CXCR4+.

15. The cell according to claim 10 wherein said retroviral packaging cell comprises a heterologous nucleic acid molecule capable of expressing a viral ligand which binds a cell surface molecule of a target cell and which optionally functions to mediate fusion of a cell membrane containing said viral ligand with said target cell.

16. The cell according to claim 1 wherein said cell is a T cell or a cell of a T cell line.

17. The cell according to claim 1 wherein said cell expresses CD86 but not CD54.

18. A recombinant retroviral packaging cell comprising a first nucleic acid molecule capable of expressing, in said packaging cell, at least one membrane associated non-viral ligand which binds a cell surface molecule of a target cell and activates or stimulates the cell into cell cycle transition.

19. The cell according to claim 18 wherein said packaging cell expresses a retroviral structural protein or accessory protein.

20. The cell according to claim 18 further comprising a retroviral vector to be packaged.
